(19) United States
(12) Patent Application Publication
(10) Pub. No.: US 2002/0102571 A1
(43) Pub. Date: Aug. 1, 2002

(54) Use of 2'-O-methyl RNA as hybridisation probe

(76) Inventors: Jane Theaker, Northwich (GB); Kay Callaghan, Northwich (GB)

Correspondence Address: PILLSBURY WINTHROP LLP, Intellectual Property Group, 1600 Tysons Boulevard, McLean, VA, 22102, US

(21) Appl. No.: 09/883,489

(22) Filed: Jun. 19, 2001

Related U.S. Application Data
(63) Continuation of application No. 09/463,324, filed on Jan. 24, 2000, now abandoned.
Application No. 09/463,324 is a 371 of international application No. PCT/GB98/02176, filed on Jul. 21, 1998.

(30) Foreign Application Priority Data
Jul. 24, 1997 (GB) 9715522.0

(51) Int. Cl. C12Q 1/68; C07H 21/04

(52) U.S. Cl. 435/6; 536/24.3

(57) ABSTRACT

Digestion-resistant probes comprising 2′-O-methyl RNA for nucleic acid hybridization assays. Their preparation and use.

This invention relates to improvements of the nucleic acid probes known as Molecular Beacons. Specifically, the use of modified nucleic acids for the synthesis of such probes has advantages in the detection of nucleic acid targets, in several applications including in situ hybridisation and homogeneous real time PCR assays.

Molecular Beacons are recently described nucleic acid probes that fluoresce upon hybridisation (Tyagi and Kramer, Nature Biotechnology 14: 303-308: 1996). The secondary structure of the probes is key and depends upon their sequence. Each probe has a target complementary portion (the loop) of about 15 or more nucleotides and additional sequences appended to each end that are complementary to each other. Finally, the probe has a fluorescent reporter molecule on one end and a fluorescence quencher at the other end. The self complementary end sequences can form stems by base pairing, thus bringing the fluorophore and quencher into close proximity and eliminating the fluorescence signal. However, in the presence of (loop) complementary target, the loop and target base pair and this “stretches out” the probe strand, thus removing the fluorophore from the quencher and allowing the reporter to fluoresce when illuminated appropriately. In this way, fluorescence is dependent upon hybridisation.

Molecular Beacons offers a number of advantages. It can be performed as a homogeneous assay. Because the “switching on” of the signal only occurs in the presence of target, there is no requirement to wash away excess unbound probe. This makes Molecular Beacons ideal for applications such as in situ hybridisation and real time PCR. It yields low background signals. In the absence of target and at an appropriate temperature, the probe self anneals and quenches fluorescence. When amplicon accumulates, some of the probe binds and is unwound, thereby generating fluorescence. In this way homogeneous (no wash, closed tube) fluorescence is produced and the low signals in the absence of target results in high ratios of signal to noise. It offers design flexibility. The use of standard stems allows rapid and reliable probe design. Furthermore, by correct design and optimisation of the stems, backgrounds at temperatures appropriate for PCR can be minimised.

For homogeneous analysis, the loop element of a Molecular Beacon should be thermodynamically favoured over the stem portion, but the stem should form readily at the assay temperature. These key requirements make it likely that long loops and stems are necessary, which has consequences for the synthesis yields and purity of Molecular Beacons. Furthermore, while long loops produce good separations between fluorophore and quencher, long stems melt over a broad temperature window leading to higher background signals and greater noise in those signals.

We have devised and now provide modified RNA probes which have a number of beneficial characteristics when used as Molecular Beacons.

Therefore in a first aspect of the present invention we provide a Molecular Beacons assay method using a 2′-O-substituted RNA probe having both a donor and a quencher species attached. The 2′-O-substituent is conveniently a methyl, propyl, butyl or allyl group. It is preferably a 2-methyl group.

The Molecular Beacons assay method is disclosed in WO-95/13399 (Public Health Research Institute of New York). This essentially comprises the detection of a pre-selected nucleic acid sequence by contacting a sample believed to contain said sequence with a Beacons probe under hybridisation conditions such that there is a detectable change in Beacons signal from the probe if the pre-selected sequence is present in the sample.

The Beacons probe is a unitary probe comprising a single stranded target complementary sequence, a stem duplex consisting of nucleotide sequences 5′ and 3′ to the target complement sequence and having a melting temperature lower than the target complementary sequence/target sequence melting temperature, and at least one label pair, each pair comprising a first label conjugated to the probe at or near the 5′ terminus of the probe and a second label conjugated to the probe at or near the terminus of the probe. Under assay conditions, hybridisation of the target complementary sequence to the target sequence leads to a chance in Beacons signal from the label pair.

The donor and acceptor species are attached to the Molecular Beacons probe in any convenient way. By way of non-limiting example either species may be attached to the 3′ terminus of the probe via controlled pore glass (CPG) based synthesis or attached to the 5′ terminus via 5′-phosphoramidite chemistry. The donor and quencher species are attached at any convenient locations on the probes, such as for example at or near the ends of the probe, preferably at the ends of the probe. Examples of convenient donor species will be apparent to the scientist of ordinary skill and include FAM, TET, JOE, HEX, ROX, BODIPY and EDANS. Convenient acceptor species include TAMRA. Pyrene butyrate, and DABCYL. Still further convenient details are found in, for example Livak et al. “PCR methods and applications, 1995, 4, 357-362: WO-95/13399 (Public Health Research Institute of New York).

Advantages of the 2′-O-substituted probes include a higher Tm when annealed to DNA target, relative to the “standard” DNA probes: this means that shorter probes can be designed to function at similar temperatures and therefore a higher level of specificity is possible—a single mismatch within a short oligonucleotide has a greater destabilising effect than when the same mismatch is in a larger hybridised region.

Secondly ‘traditional’ Molecular Beacons perform optionally in magnesium concentrations of 3.5 mM or greater. With the new 2′-O-substituted Molecular Beacons significantly lower magnesium concentrations can be utilised. This has substantial benefits for the specificity of PCR and in particular ARMS reactions. [ARMS technology is described in, for example Newton et al., 1989, Nucleic Acids Research (17) 2503-2516].

Also, annealing of 2′-O-substituted RNA to complementary 2′-O-substituted RNA targets is even more favourable than to DNA targets: this permits a shorter stem region to be used which has particular benefits for the design of effective Beacon probes. This has three functional benefits for the use of Molecular Beacons, particularly in real time PCR systems.

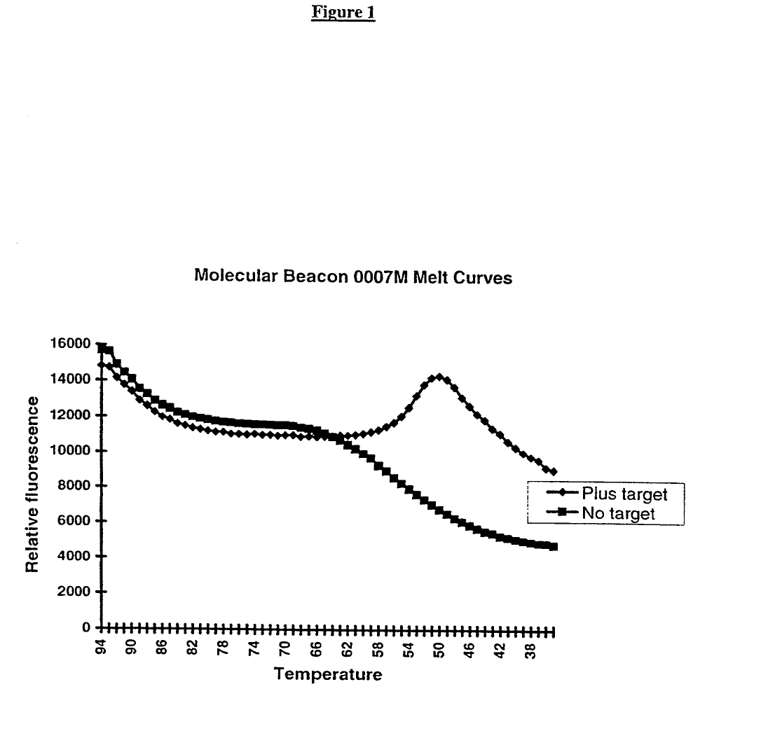

Firstly, when a Molecular Beacon is cooled from high temperature (94° C.) to low temperature (20° C.) the fluorescence changes from high (Beacon is a random coil) to low (Beacon has adopted a stem/loop formation), see FIG. 1. The temperature range of the transition from “random coil” to stem-loop structure depends upon the length of the stem sequence: longer sequences are expected to melt over a wider temperature window, possibly in a series of small steps, while shorter stems are more likely to undergo a single, rapid stem transition. This is important for the design of effective real time assays in which backgrounds should be as low as possible: the stem/loop structure should be fully formed at the temperature at which the fluorescence is measured and the probe/target interaction should also be favoured at this temperature.

Secondly, short stems have an additional advantage in that they should exhibit lower noise in positive hybridisations, due to their lower flexibility, and hence reduced ability to give a degree of fluorophore quenching.

Thirdly, at lower temperatures, the free stem sequences can “find” each other, even when the loop and the target have annealed. The hybridised duplex can be bent around and thus quenching of the fluorescence signal can occur. The consequences of this feature also favour the design of Molecular Beacons with shorter loop portions to minimise this hybrid bending, since shorter duplexes are more physically constrained and cannot bend so easily into this closed formation.

A still further advantage is that where true quantitation of PCR product is required, it is desirable to have no cleavage of dual labelled probes since the fluorescence observed in such reactions reflects the accumulation of the cleavage reactions through all the previous cycles, thus obscuring to some extent the true level of amplicon actually accumulated thus far. Many of the enzymes commonly used in PCR have an endogenous 5′-nuclease activity and may cleave the probes.

Therefore in a further aspect of the present invention we provide 2′-O-substituted RNA probes having both a donor and a quencher species attached. The 2′-O-substituent is conveniently a methyl, propyl, butyl or allyl group. It is preferably a 2-O-methyl group. These modified nucleic acids are nuclease resistant; this is important in assays where nucleases may be present such as in situ hybridisation or in real time PCR assays.

The invention further relates to diagnostic kits comprising one or more of the Beacons probes of the invention, together with appropriate buffers and other reagents, and instructions for use.

The invention will now be illustrated but not limited by reference to the following Example and Figures wherein:

FIG. 1 shows cooling curves for 2-Methyl RNA Molecular Beacon 0007M, in the presence or absence of synthetic oligonucleotide target (R297). Fluorescence readings were taken throughout the cooling range and plotted against temperature. Raw fluorescence intensity is along the Y-axis.

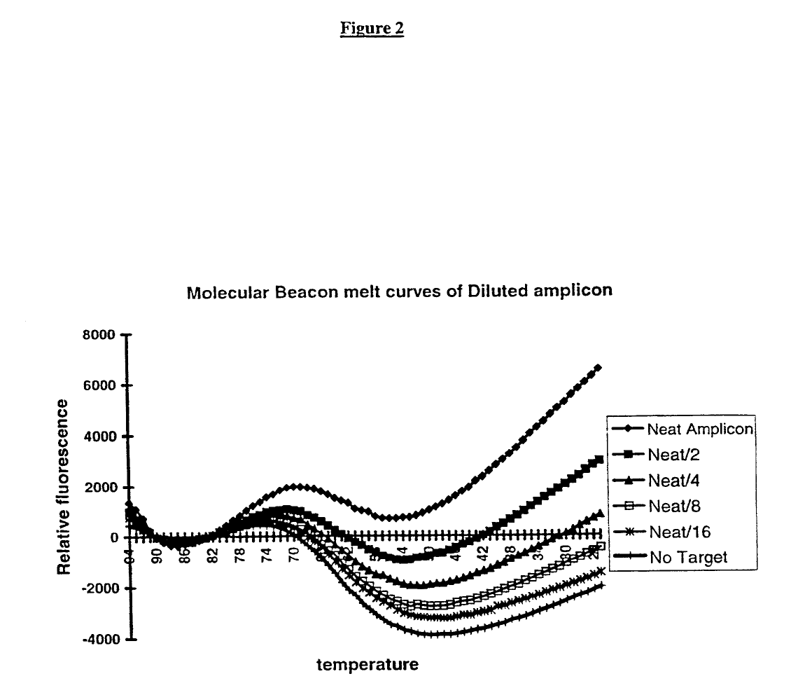

FIG. 2 shows a similar series of curves in which PCR amplicons at various concentrations from neat to ⅛ diluted were cooled in the presence of Beacon 0007M. On the X-axis temperature decreases from 94° C. to 11° C. as before, but to permit accurate tube to tube comparisons, the data are equalised to a baseline between temperatures 84° C. to 74° C. where each of the curves is flat. When the mixtures have cooled to below 50° C., the curves reach their minimum fluorescences and it is appropriate to compare them. The fluorescence value at this point rise with the quantity of target within the tube.

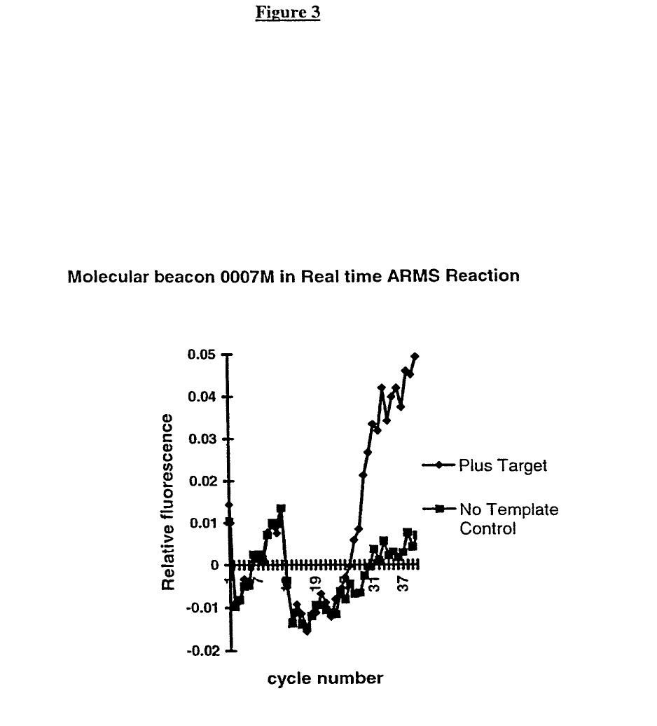

FIG. 3 illustrates the increase in Beacon fluorescence monitored at the same point in every PCR cycle (the 60° C. anneal step). Where template is included in the reaction, fluorescence increases above the background (shown by the no template control). This increase becomes significant at ˜28 cycles.

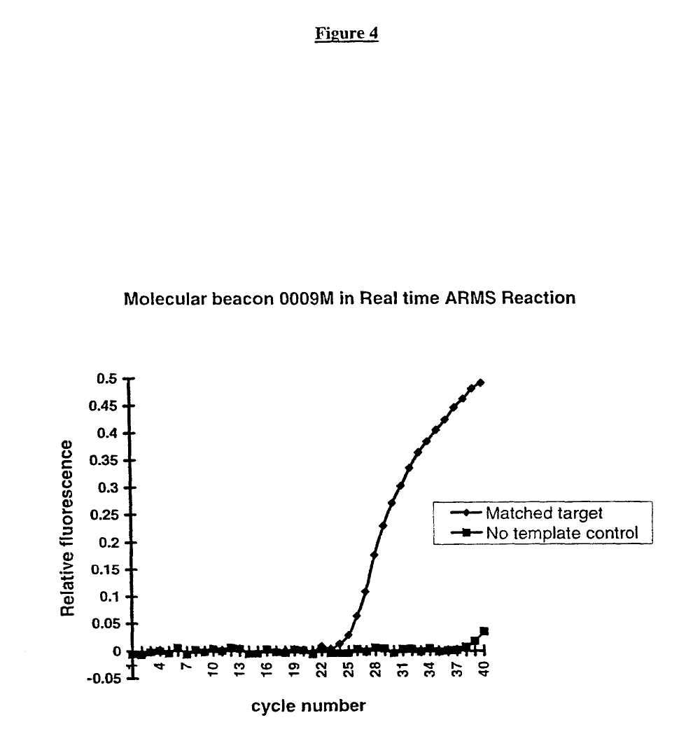

FIG. 4 shows the same reactions using Beacon 0009M in place of the previous 0007M Beacon. The fluorescence grows smoothly with less background noise and reaches substantially larger values. This reflects the improved efficiency of this Beacon due to enhanced design.

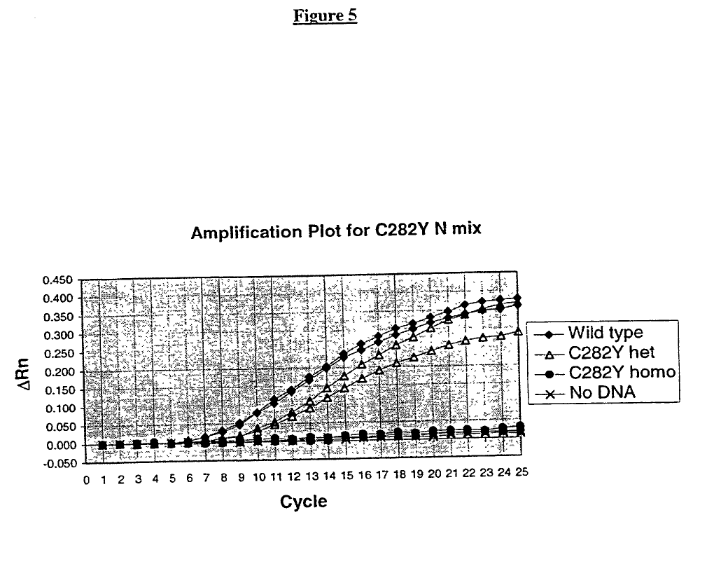

FIG. 5 shows the detection of normal alleles (N mix) of the hereditary haemochromatosis mutations C282Y het and C282Y homo, together with wild type and control sequences.

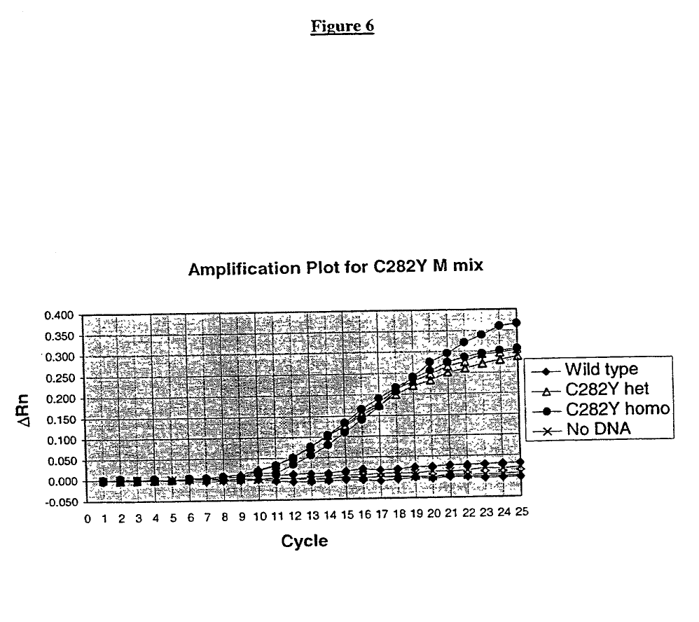

FIG. 6 shows the detection of mutant alleles (M mix) of the hereditary haemochromatosis mutations C282Y het and C282Y homo, together with wild type and control sequences.

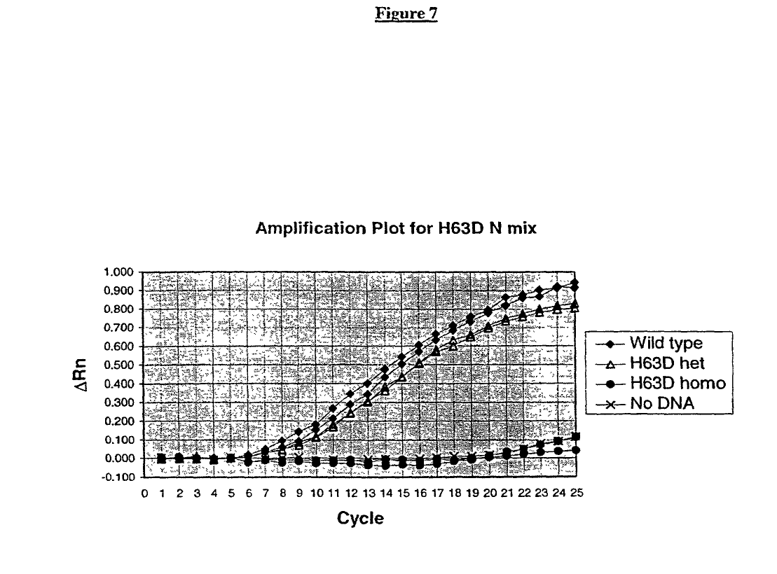

FIG. 7 shows the detection of normal alleles (N mix) of the hereditary haemochromatosis mutation H63D, together with wild type and control sequences.

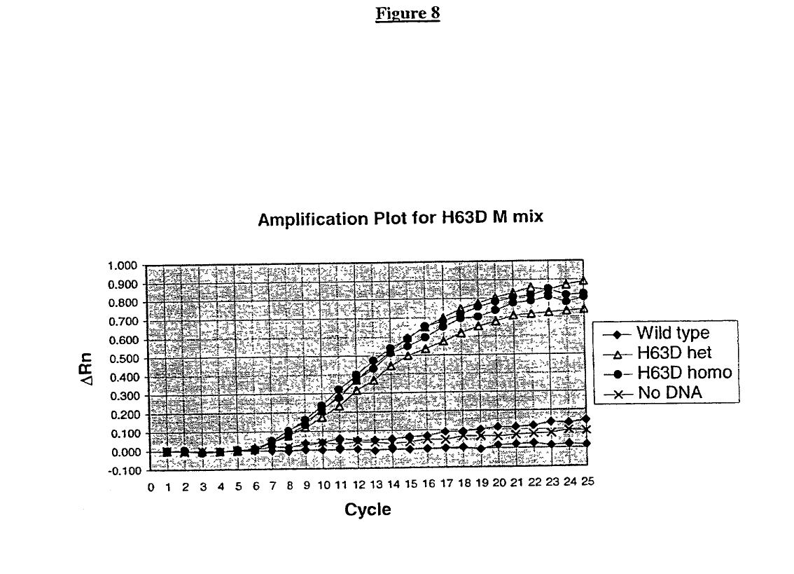

FIG. 8 shows the detection of mutant alleles (M mix) of the hereditary haemochromatosis mutation H63D, together with wild type and control sequences.

### EXAMPLE 1

Reagents

Primers and Probes

R351: CGC TGA TGA ATG TGA AAA ATC TAA

R352: AGA AGT TCC AGA TAT TGC CTG CTT

0007M (2-methylRNA Molecular Beacon): (FAM)-GCG AGC AAA AGA CCU AUU AGA CAC AGA GAA GCU CGC-(Quencher);, underlined portions indicate the self complementary stems.

R297: CTT TTG TTC TCT GTG TCT AAT ACG TCT TTT TCT GAA; synthetic target for Beacon 0007M, underlined region is the complementary portion.

Other Reagents

| | |
|---|---|
| 10 × ARMS(35) Buffer: | 100 mM Tris-HCl (pH 8.3 at 25° C). |
| | 500 mM KCl, 3.5 mM MgCl₂, 0.1% gelatin) |
| 1 mM dNTPs: | 1 mM each dNTP diluted from |
| | 10 mM stocks(Pharmacia) |
| Amplitaq Gold (5U/μl): | from Perkin-Elmer |
| ROX standard: | ROX conjugated oligonuclcotidc. |
| | 600 nM stock solution |

Melt Characteristics

Reaction Mix

400 nM Molecular Beacon 0007M in 1×ARMS (3.5) [diluted from 10-fold stock (10×ARMS)] plus dNTPs at 100 mM final, ROX standard at 60 nM and targets at various concentrations. Targets are either synthetic oligo target (R297) at 1 μM or double stranded amplicon produced by PCR with primers R351 and R352 and serially diluted.

Cycling Parameters

94° C. 2 min, then 84 steps of 15 seconds decreasing from 94° C. to 11° C. using an ABI PRISM system 7700, fluorescence readings monitored at each temperature.

Results

FIG. 1 shows the results obtained with 0007M in the presence (♦) or absence (▪) of target. The high fluorescences at elevated temperatures show the stem has not formed and the probe is essentially randomly coiled. As the temperature decreases, the fluorescence decreases in the “no target” reactions while in the presence of excess target, an increase in fluorescence is observed, peaking at around 56° C. The subsequent reduction in this fluorescence reflects the high affinity of the stems for each other and at lower temperatures, the probe target duplex can be bent permitting stems to form and fluorescence to switch off.

FIG. 2 shows a similar series of curves with varying quantities of double-stranded amplicon: a 2-fold serial dilution from “neat” to 1/16 and a negative control. The data have been equalised for fluorescence between 88° C. and 74° C. (where each line is flat) to allow direct comparison between tubes. There is a clear difference between positive and negative samples and the dilution series of target numbers is clearly reflected in the relative fluorescences, particularly at lower temperatures. There are two further observations to be drawn from this data:

1. the larger amplicon target is less prone to subsequent bending and closure than the oligonucleotide target:

2. the target was heat denatured only once and it might have been expected that the “other” strand would displace the probe from its binding site. This was not the case since fluorescences remained substantial throughout the cooling.

Amplification Reactions

Reaction Mix

1×ARMS (3.5), buffer added to 100 μM dNTPs, 500 nM each primer (R351, R352), 60 nM ROX standard, 400 nM Beacon 0007M, 2 Units Amplitaq Gold (per 50 μl reaction), 5 μl (50 ng) human genomic DNA.

Cycling Conditions

The reactions were cycled and fluorescence read using the conditions below. 94° C. for 20 min to activate the Amplitaq Gold. Followed by 40 cycles of: 94° C. for 41 s. 60° C. for 42 s. 72° C. for 52 s. Fluorescence data was collected and analysed for the 60° C. anneal step.

Results

The output from an amplification reaction as described above is shown in FIG. 3. There is a clear difference between positive and negative samples using this system. We anticipate that the probe design and reaction conditions may be further optimised, thus yielding increased fluorescences, whilst minimising the signal noise and enhancing the sensitivity of the technique.

### EXAMPLE 2

In this Example we disclose a further, improved Molecular Beacon. In this the loop portion of the probe was enlarged to ensure strong binding to target and good physical separation of fluorophore from quencher. In addition, the stem was decreased in size to a CGCG tetramer which has the multiple benefits of short length to minimise the folding back of the bound duplex, high Tm due to its particular sequence composition, 5′-terminal C adjacent to the fluorophore which avoids possible quenching by the G, as present in probe 0007M.

The improved Beacon is 0009M: (FAM)-CGC GGA AAA ASA CCU AUU AGA CAC AGA GAA CAC GCG-(Quencher). Underlined portions are the stem. All bases are 2′-O-methyl RNA.

### EXAMPLE 3

Detection of Hereditary Haemochromatosis mutations using 2′-O-methyl RNA molecular beacons

1) C282Y ARMS Test

Materials and Methods

Two mixes were made up, the first (N) to detect the normal (wild type) allele and the second (M) to detect the mutant allele. The mixes differ only by the 3′ base of the ARMS primer. Final concentrations of mix components are shown below:

| C282Y N mix | C282Y M mix |
|---|---|
| 1x Beacons buffer | 1x Beacons buffer |
| 100 mM dNTPs | 100 mM dNTPs |
| 1x Passive Reference (60 nM ROX) | 1x Passive Reference (60 nM |
| | ROX) |
| 0.5 mM common forward | 0.5 mM common forward |
| primer (R279-97) | primer (R279-97) |
| 0.5 mM reverse ARMS normal | 0.5 mM reverse ARMS normal |
| primer (R280-97) | primer (R281-97) |
| 0.4 mM molecular beacon (0037M) | 0.4 mM molecular beacon |
| | (0037M) |
| 1 unit AmpliTaq Gold enzyme | 1 unit AmpliTaq Gold enzyme |

Composition of 1×Beacons buffer:

50 mM KCl

10 mM Tris (pH 8.3)

3.5 mM MgCl₂

0.01% (w/v) gelatin

Primer/Beacon Sequences (5′→3′):

| | |
|---|---|
| R279-97 | AACTGCCTCCTTTGGTGAAGCTGACACA |
| R280-97 | TGATCCAGGCCTGGGTGCTCCACCTGAC |
| R281-97 | TGATCCAGGCCTGGGTGCTCCACCTGAT |
| 0037M | (FAM)-CGCGAGUUCGAACCUAAAGACGUAUUGCCCAACGCG-(Quencher) |

The mixes were dispensed into 20 μl aliquots in optical tubes. 5 μl of genomic DNA (prepared from blood by alkali lysis and diluted ⅕ in Water) was added to duplicate aliquots of both mixes. Examples of wild type, heterozygous mutant and homozygous mutant samples were used.

The tubes were place in a thermal cycler (Applied Biosystems 7700) and the following PCR program was run:

20 minutes at 94° C. followed by

20 cycles of

45 seconds at 94° C.

45 seconds at 60° C.

then 25 cycles of

45seconds at 94° C.

45 seconds at 40° C.

The results are shown in FIG. 5 and FIG. 6.

The results above demonstrate that the N mix only amplifies genomic DNA which contains the wild type allele (solid diamonds and clear triangles) and the M mix only amplifies genomic DNA which contains the mutant allele (clear triangles and solid circles). Amplification does not occur in either mix in the absence of genomic DNA (Xs).

2) H63D ARMS Test

Materials and Methods

Two mixes were made up, the first (N) to detect the normal (wild type) allele and the second (M) to detect the mutant allele. The mixes differ only by the 3′ base of the ARMS primer. All primers in the H63D test have the same 26mer non-homologous tail sequence at their 5′-end. Final concentrations of mix components are shown below:

| H63D N mix | H63D M mix |
|---|---|
| 1x Beacons buffer | 1x Beacons buffer |
| 100 mM dNTPs | 100 mM dNTPs |
| 1x Passive Reference (60 nM ROX) | 1x Passive Reference (60 nM |
| | ROX) |
| 0.5 mM common forward primer | 0.5 mM common forward primer |
| (R369-98) | (R369-98) |
| 0.5 mM reverse ARMS normal | 0.5 mM reverse ARMS normal |
| primer (R370-99) | primer (R371-98) |
| 0.4 mM molecular beacon | 0.4 mM molecular beacon |
| (MB026-98) | (MB02-98) |
| 1 unit AmpliTaq Gold enzyme | 1 unit AmpliTaq Gold enzyme |

Composition of 1×Beacons buffer

50 mM KCl

10 mM Tris (pH 8.3)

3.5 mM MgCl₂

0.01% (w/v) gelatin

Prime/Beacon Sequences (5′→3′)

R369-98

GCGTACTAGCGTACCACGTGTCGACTTCCTACTACACATGGTTAAGGCCTG

R370-98

GCGTACTAGCGTACCACGTGTCGACTGGGCTCCACACGGCGACTCTCAAG

R371-98

GCGTACTAGCGTACCACGTGTCGACTGGGCTCCACACGGCGACTCTCAAC

MB026-98 (FAM)-CGCGGGAUGACCAGCUGUUCGUGUUCUACGCG-(Quencher)

The mixes were dispensed into 20 μl aliquots in optical tubes. 5 μl of genomic DNA (prepared from blood using the Gentra PureGene kit and diluted in water to 10 ng/μl) was added to duplicate aliquots of both mixes. Examples of wild type, heterozygous mutant and homozygous mutant samples were used.

The tubes were placed in a thermal cycler (Applied Biosystems 7700) and the following PCR program was run:

20 minutes at 94° C. followed by

20 cycles of

45 seconds at 94° C.

45 seconds at 60° C.

then 25 cycles of

45 seconds at 94° C.

45 seconds at 40° C.

The results are shown in FIGS. 7 and 8.

Again, the results demonstrate that the N mix only amplifies genomic DNA containing the wild type allele (solid diamonds and clear triangles) and the M mix only amplifies genomic DNA containing the mutant allele (clear triangles and solid circles). Amplification does not occur in either mix in the absence of genomic DNA (Xs).

The amplification protocol was identical to the run shown in FIG. 3. The results from this amplification are shown in FIG. 4.

We claim:

1. A nuclease resistant probe comprising 2′-O-substituted RNA and having donor and acceptor species attached at or near its termini.

2. A probe as claimed in claim 1 and further comprising a stem duplex consisting of nucleotide sequences 5′ and 3′ to a target complementary sequence.

3. A probe as claimed in claim 2 wherein the stem duplex is of 3-6 bases in length.

4. A probe as claimed in any one of claims 1-3 wherein the 2-O-substituent is an alkyl group.

5. A probe as claimed in claim 3 wherein the alkyl group is methyl.

6. A diagnostic assay which comprises the use of a probe as claimed in any one of the previous claims.

7. A diagnostic assay as claimed in claim 6 and being a real time assay.

8. A diagnostic kit comprising one or more probes as claimed in any one of the previous claims together with one or more of appropriate buffers, reagents and instructions for use.


SEQUENCE LISTING (14 sequences)

SEQ ID NO: 1 - DNA, 24 bp, Artificial Sequence
  Description of Artificial Sequence synthetic oligonucleotide
cgctgatgaatgtgaaaaatctaa

SEQ ID NO: 2 - DNA, 24 bp, Artificial Sequence
  Description of Artificial Sequence synthetic oligonucleotide
agaagttccagatattgcctgctt

SEQ ID NO: 3 - RNA, 36 nt, Artificial Sequence
  Description of Artificial Sequence synthetic oligonucleotide
gcgagcaaaagaccuauuagacacagagaagcucgc

SEQ ID NO: 4 - DNA, 36 bp, Artificial Sequence
  Description of Artificial Sequence synthetic oligonucleotide
cttttgttctctgtgtctaataggtctttttctgaa

SEQ ID NO: 5 - DNA, 32 bp, Artificial Sequence
  Description of Artificial Sequence synthetic oligonucleotide
cgcggaaaaaaccaagacacagagaacacgcg

SEQ ID NO: 6 - DNA, 28 bp, Artificial Sequence
  Description of Artificial Sequence synthetic oligonucleotide
aagtgcctcctttggtgaagctgacaca

SEQ ID NO: 7 - DNA, 28 bp, Artificial Sequence
  Description of Artificial Sequence synthetic oligonucleotide
tgatccaggcctgggtgctccacctgac

SEQ ID NO: 8 - DNA, 28 bp, Artificial Sequence
  Description of Artificial Sequence synthetic oligonucleotide
tgatccaggcctgggtgctccacctgat

SEQ ID NO: 9 - RNA, 36 nt, Artificial Sequence
  Description of Artificial Sequence synthetic oligonucleotide
cgcgaguucgaaccuaaagacguauugcccaacgcg

SEQ ID NO: 10 - DNA, 51 bp, Artificial Sequence
  Description of Artificial Sequence synthetic oligonucleotide
gcgtactagcgtaccacgtgtcgacttcctactacacatggttaaggcctg

SEQ ID NO: 11 - DNA, 50 bp, Artificial Sequence
  Description of Artificial Sequence synthetic oligonucleotide
gcgtactagcgtaccacgtgtcgactgggctccacacggcgactctcaag

SEQ ID NO: 12 - DNA, 50 bp, Artificial Sequence
  Description of Artificial Sequence synthetic oligonucleotide
gcgtactagcgtaccacgtgtcgactgggctccacacggcgactctcaac

SEQ ID NO: 13 - RNA, 32 nt, Artificial Sequence
  Description of Artificial Sequence synthetic oligonucleotide
cgcgggaugaccagcuguucguguucuacgcg

SEQ ID NO: 14 - RNA, 36 nt, Artificial Sequence
  Description of Artificial Sequence synthetic oligonucleotide
cgcggaaaaagaccuauuagacacagagaacacgcg